(19) United States
(12) Patent Application Publication
(10) Pub. No.: US 2006/0294606 A1
(43) Pub. Date: Dec. 28, 2006

(54) Tn5 transposase-mediated transgenesis

(76) Inventors: Istefo Moisyadi, Honolulu, HI (US); Ryuzo Yanagimachi, Honolulu, HI (US); Ryota Suganuma, Fukushima (JP)

Correspondence Address: GODBEY GRIFFITHS REISS, 1001 BISHOP STREET, 2300 PAUAHI TOWER, HONOLULU, HI, 96813, US

(21) Appl. No.: 11/127,685

(22) Filed: May 11, 2005

Related U.S. Application Data
(60) Provisional application No. 60/572,701, filed on May 18, 2004.

(51) Int. Cl. A01K 67/027

(52) U.S. Cl. 800/14; 800/15; 800/16; 800/17; 800/18; 800/20

(57) ABSTRACT

The present invention relates to methods for obtaining transgenic embryos, animals, and plants. Also encompassed in the present invention are transposase-mediated trangenesis methods including transposase-mediated intracytoplasmic sperm injection (TN:ICSI), transposase-mediated intracytoplasmic round spermatid injection (TN:ROSI), and transposase-mediated in vitro fertilization (TN:IVF).

### RELATED APPLICATION

This application claims the benefit of U.S. Provisional Application No. 60/572,701, filed on May 18, 2004. The entire teaching of the above application is incorporated herein by reference.

### BACKGROUND OF THE INVENTION

The generation of transgenic embryos is essential in both basic and applied genetic research and in commercial applications. Transgenesis relies on the integration of exogenous nucleic acid into a host cell. Integration of the nucleic acid can be characterized as either passive or active. In passive transgenesis, the nucleic acid is integrated into the genome of the host cell via DNA repair mechanisms and is a low frequency event. In contrast, active transgenesis does not rely solely on host DNA repair mechanisms and occurs at a higher frequency than passive transgenesis. Currently, active transgenesis methods are limited. Thus, there is a need for improved active transgenesis methods.

### SUMMARY OF THE INVENTION

The present invention relates to active transgenesis methods comprising the use of transposases. The transgenesis methods described herein are advantageous for generating transgenic embryos and animals for research, therapeutic, and commercial applications utilizing fewer oocytes than conventional methods.

In one embodiment, the present invention is directed to a method for obtaining a transgenic embryo comprising incubating a mixture of a transposable exogenous nucleic acid and a transposase or a hyperactive mutant of said transposase or a nucleic acid encoding said transposase; contacting said mixture with a sperm; and introducing said mixture contacted with said sperm into an oocyte to form a transgenic embryo, whereby said transposase catalyzes integration of said transposable exogenous nucleic acid into the genome of said embryo.

Exogenous nucleic acids, sperm, pollen, male gametes, sperm heads, oocytes, ova, and female gametes obtained from any suitable organism including vertebrates, invertebrates, plants, mammals, fish, amphibians, reptiles, birds, rodents, cows, pigs, sheep, goats, and horses are useful in the invention. The present invention encompasses transposable exogenous nucleic acids that are flanked by nucleic acid sequences to form an inverted repeat sequence recognized by a transposase. Inverted repeat sequences useful in the present invention include nucleic acid sequences comprising SEQ ID NO: 1, SEQ ID NO: 2, and SEQ ID NO:3. The transposable exogenous nucleic acid may contain more than one transgene and/or more than one transposable exogenous sequence. Prokaryotic and eukaryotic transposases are useful in the present invention. The present invention encompasses transposases comprising the amino acid sequence having at least 80% sequence identity to SEQ ID NO: 4; the amino acid sequence comprising SEQ ID NO: 4; and the amino acid sequence encoded by the nucleic acid sequences comprising SEQ ID NO: 5 or SEQ ID NO: 6.

The present invention encompasses incubating the transposable exogenous nucleic acid and the transposase or nucleic acid encoding the transposase for about five minutes to about five hours; for about twenty minutes to about two hours; or about thirty minutes.

The present invention encompasses contacting the transposable exogenous nucleic acid, the transposase or nucleic acid encoding the transposase, and the sperm for about thirty seconds to about five minutes; or for about two minutes.

A further aspect of the present invention relates to a method for generating a transgenic embryo comprising incubating a mixture of a transposable exogenous nucleic acid and a transposase or a hyperactive mutant of said transposase or a nucleic acid encoding said transposase; and introducing said mixture into an in vitro fertilized (IVF) oocyte to form a transgenic embryo, whereby said transposase catalyzes integration of said transposable exogenous nucleic acid into the genome of said embryo.

In another aspect, the present invention is directed toward a method of obtaining a transgenic embryo comprising incubating a mixture of a transposable exogenous nucleic acid and a transposase or a hyperactive mutant of said transposase or a nucleic acid encoding said transposase; contacting said mixture with a round spermatid; and introducing said mixture contacted with said spermatid into an artificially activated oocyte to form a transgenic embryo, whereby said transposase catalyzes integration of said transposable exogenous nucleic acid into the genome of said embryo.

The invention encompasses implantation of the transgenic embryo into a suitable surrogate mother and allowing the transgenic embryo to develop into a transgenic offspring. Reagents useful in the invention include unfertilized metaphase II stage oocytes, in vitro fertilized (IVF) oocytes, artificially activated oocytes, ova, spermatozoa, spermatids, sperm heads, pollen, demembranated sperm, and membrane disrupted sperm. Methods for introducing components of the invention such as the transposable exogenous nucleic acid, transposase, and sperm head into an oocyte include microinjection, intracytoplasmic sperm injection (ICSI), pronuclear microinjection, particle bombardment, electroporation, and lipid vesicle transfection.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a schematic diagram of the DNA vector construct for the transposable nucleic acid and the formation of a transposome.

FIG. 1B is a schematic diagram of the transgenesis method.

FIG. 2A is a schematic diagram of a transposome.

FIG. 2B is an ultraviolet light illuminated-photograph of a transgenic mouse (t) and a nontransgenic control mouse (c) generated by TN:ICSI.

FIG. 2C is a picture of a DNA gel electrophoresis demonstrating PCR analysis of transgenic animals obtained by TN:ICSI.

FIG. 2D is a Southern blot hybridization analysis of TN:ICSI transgenic animals.

FIG. 3A is a Southern blot hybridization analysis of TN:ROSI transgenic animals.

FIG. 3B is an ultraviolet light illuminated-photograph of a transgenic mouse (t) and a nontransgenic control mouse (c) generated by TN:ROSI.

FIG. 4 is a histogram comparing the efficiency of transgenesis methods.

FIG. 5 is a diagram illustrating the amino acid sequence of a hyperactive Tn5 transposase mutant (*Tn5p) (SEQ ID NO: 4).

FIG. 6 is a diagram illustrating the nucleotide sequence encoding a hyperactive Tn5 transposase mutant (*Tn5p) (SEQ ID NO: 5).

FIG. 7 is a diagram illustrating the mammalian codon biased nucleotide sequence encoding a hyperactive Tn5 transposase mutant (*Tn5p) (SEQ ID NO: 6).

FIG. 8 is a diagram illustrating the nucleotide sequence of the mosaic end sequence (SEQ ID NO: 1), outside end sequence (SEQ ID NO: 2), and inside end sequence (SEQ ID NO: 3) that is bound by Tn5 transposases.

### DETAILED DESCRIPTION OF THE INVENTION

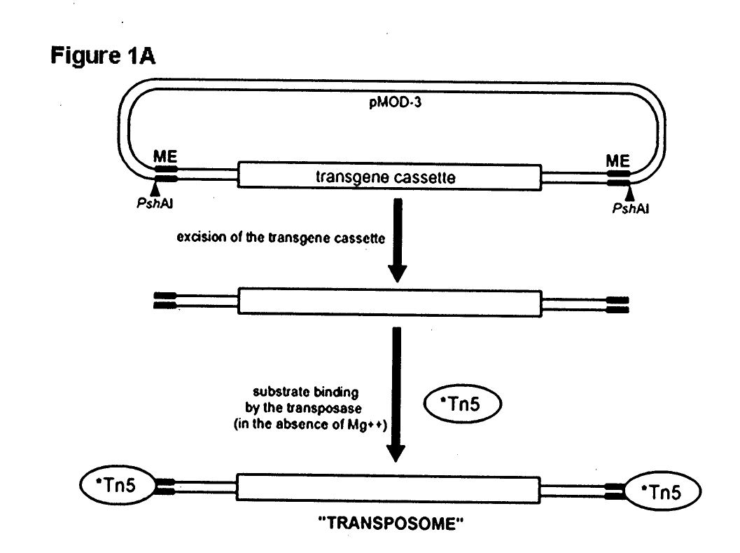

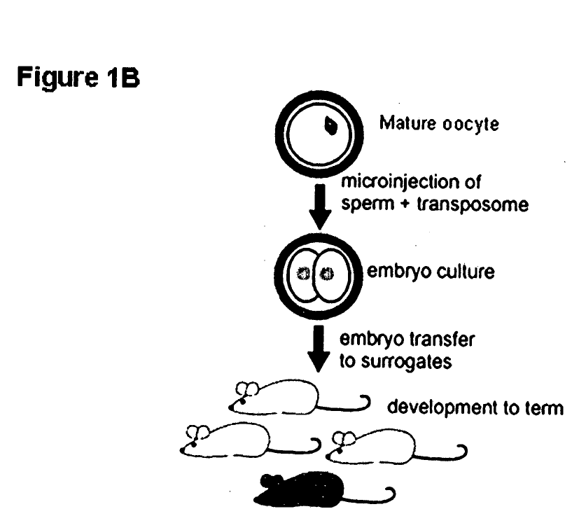

The present invention relates to the Applicants' discovery that transgenic animals can be produced by microinjection of a transposable exogenous nucleic acid, a hyperactive Tn5 transposase mutant, and a sperm head into the cytoplasm of an unfertilized metaphase II oocyte to form a transgenic embryo, whereby the transposase catalyzes integration of the transposable exogenous nucleic acid into the genome of the transgenic embryo; implanting the transgenic embryo into a surrogate mother and allowing the transgenic embryo to develop into a transgenic offspring (See FIG. 1B). Transgenic animals and plants have many uses including genetic research, gene therapy, crop and animal improvement, and producing therapeutic and non-therapeutic molecules. The present invention provides methods for generating transgenic embryos, animals, and plants. The present invention encompasses transposase-mediated trangenesis methods including transposase-mediated intracytoplasmic sperm injection (TN:ICSI), transposase-mediated intracytoplasmic round spermatid injection (TN:ROSI), and transposase-mediated in vitro fertilization (TN:IVF). The use of transposase-like enzymes to catalyze the integration of the exogenous nucleic acid into the genome of the transgenic embryo is also comprehended.

In one embodiment, the present invention is a method for obtaining a transgenic embryo comprising incubating a mixture of a transposable exogenous nucleic acid and a transposase or a hyperactive mutant of said transposase or a nucleic acid encoding said transposase; contacting said mixture with a sperm; and introducing said mixture contacted with said sperm into an unfertilized oocyte to form a transgenic embryo, whereby said transposase catalyzes integration of said transposable exogenous nucleic acid into the genome of said transgenic embryo. The method may further comprise implanting the transgenic embryo into a surrogate mother and allowing the embryo to develop into a live transgenic animal or plant. As used herein, this method can be referred to as transposase-mediated intracytoplasmic sperm injection (TN:ICSI).

Transposases are encoded by transposons and are well known to one skilled in the art. A transposase, as used herein, is an enzyme which catalyzes the transposition of a transposable nucleic acid sequence into the genome of a cell. Transposases and transposable DNA elements, referred to as transposons, have been discovered in almost all organisms. The genetic structures and transposition mechanisms of various transposons are summarized in the art, for example in “Transposable Genetic Elements” in “The Encyclopedia of Molecular Biology,” Kendrew and Lawrence, Eds., Blackwell Science, Ltd., Oxford (1994) and in “Mobile DNA II,” Craig, Gellert, and Lambowitz, Eds., American Society of Microbiology, Washington, D.C. (2002), which are incorporated herein by reference.

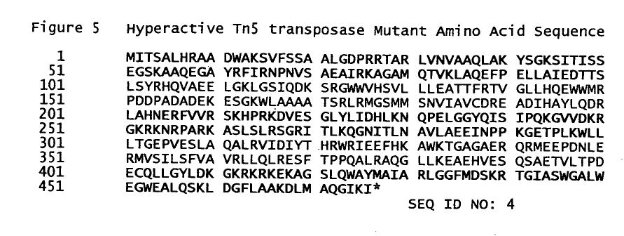

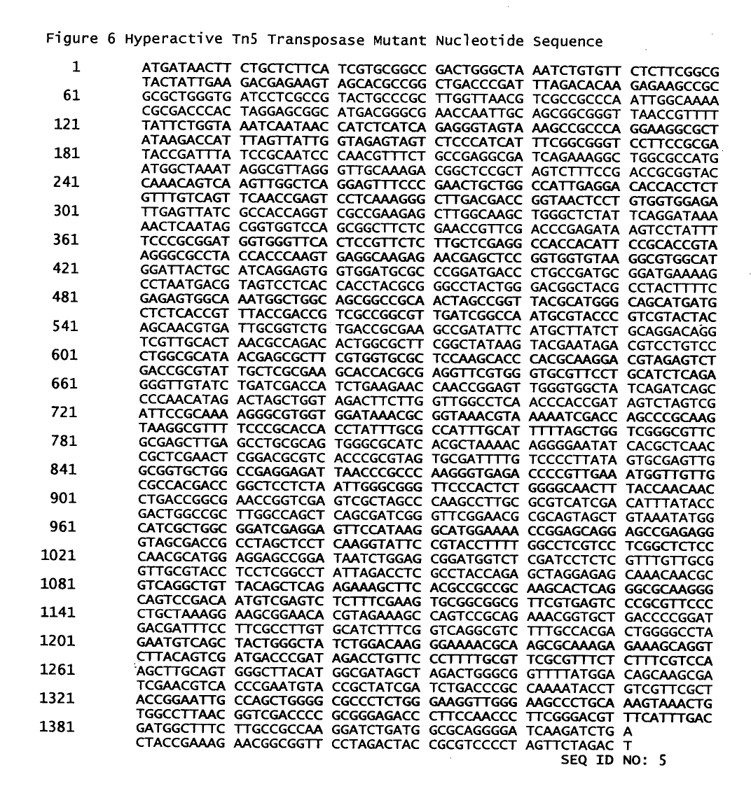

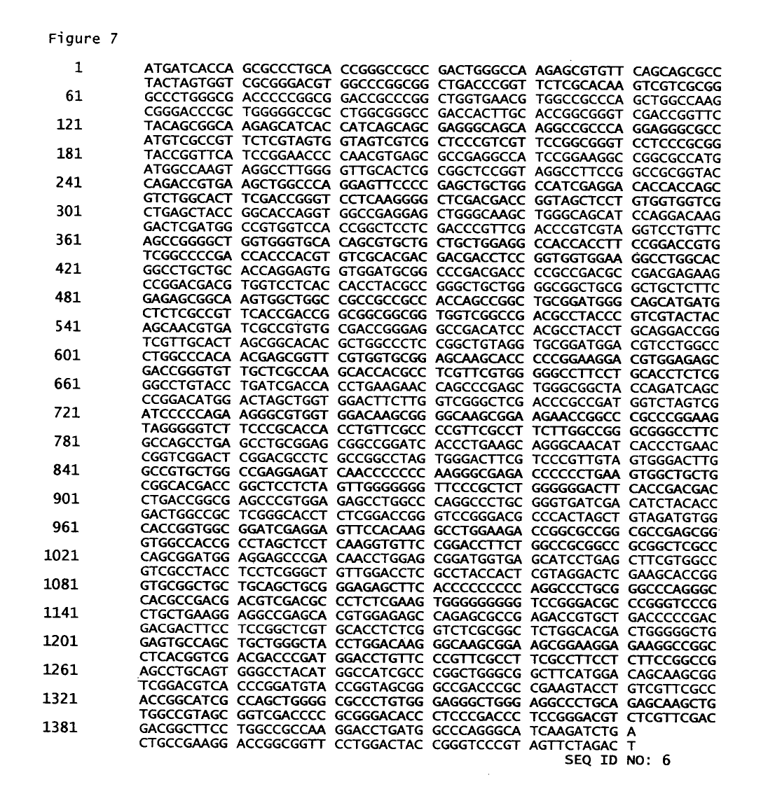

Transposases useful in this invention include prokaryotic and eukaryotic transposases and hyperactive mutants thereof. Transposases useful in the invention contain critical DDE residues (aspartate, aspartate, glutamate) that chelate two magnesium (Mg²⁺) ions, which are essential for catalysis (in “Mobile DNA II,” Craig, Gellert, and Lambowitz, Eds., American Society of Microbiology, Washington, D.C. (2002). However, transposase-like integration enzymes and transposases, which utilize different mechanisms, may be useful in the present invention. In a particular embodiment, a hyperactive Tn5 transposase mutant is used. As defined herein, a hyperactive transposase mutant is a transposase mutant that has a higher catalytic activity than the wild type transposase. In another embodiment, the transposase comprises the amino acid sequence having at least 80% identity to SEQ ID NO: 4. In a preferred embodiment, the transposase comprises the amino acid sequence of SEQ ID NO: 4 (See FIG. 5). Nucleic acid sequences encoding transposases are also useful in the present invention. Nucleic acids include deoxyribonucleic acid (DNA) and ribonucleic acid (RNA). In a particular embodiment, the transposase is encoded by the nucleic acid sequence comprising SEQ ID NO: 5 (see FIG. 6). Codon biased nucleic acid sequences encoding transposases are also useful in the present invention. As defined herein, a codon biased sequence refers to a nucleic acid sequence that accounts for the codon bias of a particular organism to encode the same amino acid sequence of a transposase. The use of codon biased sequences may improve the expression of the transposase protein in the respective organism. For example, the nucleic acid sequence comprising SEQ ID NO: 6 (See FIG. 7) is the mammalian codon bias sequence for a hyperactive Tn5 transposase amino acid sequence (SEQ ID NO:4). In another particular embodiment, the transposase of the present invention is encoded by the nucleic acid sequence comprising SEQ ID NO: 6.

Exogenous nucleic acid for use in the present invention can be derived from any source including but not limited to vertebrates, invertebrates, plants, mammals, fish, amphibians, reptiles, birds, rodents, cows, pigs, sheep, goats, and horses. Exogenous nucleic acid, as used herein, refers to any nucleic acid external to a cell. Exogenous nucleic acids can comprise any nucleic acid sequence of interest including but not limited to: genes; gene fragments; antisense nucleic acids; ribozymes; double stranded RNA or small interfering ribonucleic acid (si RNA); RNA; structural genes; reporter genes; chemically-modified nucleic acids; and nucleic acids which alter, enhance, decrease, or maintain gene function or provide new, useful, or desirable effects.

Gene, as used herein, refers to a nucleic acid sequence comprising regulatory and coding sequences necessary for the generation of ribonucleic acids and/or polypeptides. The term structural gene refers to a nucleic acid that encodes a biologically active protein or ribonucleic acid but excludes the regulatory sequences. Regulatory sequences (or regulatory elements) comprise nucleic acid sequences that control gene expression and include promoters, enhancers, or polyadenylation signals. For example, tissue-specific, inducible, and constitutive promoters can be used in the present invention to control spatial and temporal gene expression. Regulatory elements and their uses are well known in the art.

One advantage of the present invention over retroviral transgenesis is the size of the exogenous nucleic acid that can be used. The present invention can utilize nucleic acids in a size range from about 50 base pairs (bp) to about 500 mega base pairs (mb). Exogenous nucleic acids useful in the invention may contain one or more transgenes. As defined herein, transgenes are genes of interest that become integrated into the genome of a cell utilizing methods of the present invention.

Transposases are enzymes that catalyze the insertion or integration of transposable nucleic acids into the genome by binding to specific nucleotide end sequences that form an inverted repeat sequence flanking the transposable nucleic acid. Transposition, as used herein, refers to the integration or insertion of transposable nucleic acids into the genome of a cell. Transposition is well known to one of skill in the art. Inverted repeat sequences, as used herein, refer to a nucleotide sequence that flanks the exogenous nucleic acid in opposite orientations (See the mosaic end (ME) sequences in FIG. 1A).

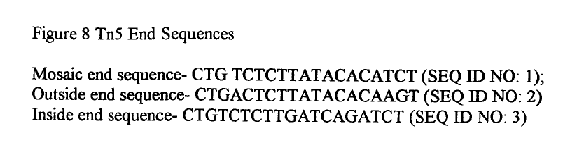

In the present invention, the transposable exogenous nucleic acid encompasses end sequences that flank the exogenous nucleic acid and form an inverted repeat that is recognized by a transposase. End sequences have a cognate transposase that binds to them and confer the ability of the nucleic acid sequence between the end sequences to undergo transposition. It will be understood by one skilled in the art that the use of mutated end sequences and/or transposases that bind to them is within the scope of the disclosed invention. A diagrammatic representation of a transposable exogenous nucleic acid is the middle structure in FIG. 1A. FIG. 1A also illustrates end sequences (e.g. SEQ ID NO: 1) bound by a specific transposase (e.g. hyperactive Tn5 transposase mutants). Molecular biological methods for constructing transposable exogenous nucleic acids are described herein and well known to one of ordinary skill in the art. In a particular embodiment, the transposable exogenous nucleic acid comprises an exogenous nucleic acid flanked by at least two 19 base pair (bp) end sequences that form an inverted repeat that is specifically recognized by the transposase. It will be understood by one skilled in the art that the end sequences need only flank the exogenous nucleic acid sequence and not be located at the termini of a nucleic acid sequence. The present invention encompasses exogenous nucleic acids which contain one or more transposable nucleic acid sequences. In a particular embodiment, the end sequence is selected from a group consisting of SEQ ID NO: 1, SEQ ID NO: 2, and SEQ ID NO: 3 (See FIG. 8). As defined herein, end sequences are also referred to as mosaic end, outside end, and inside end sequences. End sequences of use in the present invention include those described in the Examples, Reznikoff (Molecular Microbiology 47, 1199-1206 (2003)), and in references contained therein and are well known to one of skill in the art. In a particular embodiment, the end sequence is recognized by a hyperactive Tn5 transposase.

Incubating, as used herein, refers to the mixing or contacting of components of the present invention together for a suitable length of time. For example, incubating the transposable exogenous nucleic acid and the transposase or nucleic acid encoding the transposase. In one embodiment, the transposable exogenous nucleic acid and the transposase or nucleic acid encoding the transposase are incubated for about five minutes to about five hours. In another embodiment, the transposable exogenous nucleic acid and the transposase or nucleic acid encoding the transposase are incubated for about twenty minutes to about two hours. In a particular embodiment, the transposable exogenous nucleic acid and the transposase or nucleic acid encoding the transposase are incubated for about thirty minutes. The suitable time for incubation of a transposase and the nucleic acid is defined as the time it takes for the transposase to bind to the transposable nucleic acid, forming a transposome. A transposome, as used herein, refers to a complex comprising a transposase bound to a transposable nucleic acid. More specifically, the transposome comprises the transposase bound to the end sequences of the transposable exogenous nucleic acid (See FIG. 1A). A suitable incubation time may vary for different transposases, but optimizing the incubation time is routine for one of ordinary skill in the art.

The ratio of transposase to nucleic acid should be sufficient for the transposase to bind to each end sequence of the transposable nucleic acid. The molar ratio of the transposase to transposable exogenous nucleic acid useful in the present invention is about 2:1 to about 20:1; or about 5:1 to about 10:1; or about 7:1 at 37° C. Optimization of the transposase to transposable exogenous nucleic acid is well within the abilities of one of ordinary skill in the art.

Contacting, as used herein, refers to the mixing, incubating, or bringing together of components of the present invention. For example, contacting the transposable exogenous nucleic acid and the transposase or nucleic acid encoding the transposase with a sperm, sperm head, spermatid, or pollen (plants) for a suitable length of time. A sperm, as used herein, refers to a fresh sperm. Sperm head, as used herein, refers to a sperm without its tail. Spermatid, as defined herein, refers to a germ cell meiosis product that is haploid but has not yet undergone the metamorphosis of replacing the somatic histones for the protamines and is round and lacks the acrosome of the mature sperm. Spermatid, as used herein, can refer to an immature sperm. Spermatid and round spermatid are used interchangeably in the present invention. The role of the sperm, spermatid, sperm head, or male gamete during fertilization involves the transfer of a haploid genome to the resultant zygote. The oocyte or female gamete provides another haploid genome which upon fertilization with the male gamete (or introduction of the male gamete in the present invention) generates a diploid zygote.

In one embodiment, the transposable exogenous nucleic acid and the transposase or nucleic acid encoding the transposase are contacted with a sperm, sperm head, or round spermatid for about thirty seconds to about five minutes. In a particular embodiment, the transposable exogenous nucleic acid and the transposase or nucleic acid encoding the transposase are contacted with a sperm, sperm head, spermatid, or round spermatid for about two minutes.

Incubating and contacting the various components of the invention, as described herein, can be carried out separately or simultaneously.

Sperm (or the male gamete of an organism) useful in the present invention can be obtained from any suitable organism and includes but is not limited to membrane-disrupted, demembranated, and fresh sperm. Pollen, as used herein, is the sperm equivalent (male gamete) of plants for fertilizing a plant ovum, the oocyte equivalent (female gamete). Methods of obtaining membrane-disrupted, demembranated, and fresh sperm are described herein and known in the art (See U.S. Pat. No: 6,376,743).

The incubation and contacting steps in the present invention should be done in the absence of magnesium ions, if one does not want the transposase to be active before introducing the transposome/sperm head into the oocyte. It will be known to one of ordinary skill in the art that when one incubates and contacts the transposable exogenous nucleic acid, the transposase or the nucleic acid encoding the transposase, and the sperm head, magnesium ion-free conditions are not necessary, unless desired.

Oocytes useful in the invention may be from any organism including vertebrates, invertebrates, plants, mammals, amphibians, reptiles, birds, rodents, cows, pigs, sheep, goats, fish, and horses. Plant ova are useful in the present invention. In one embodiment, oocytes are unfertilized metaphase II oocytes. Methods for harvesting oocytes at the appropriate stage are described herein and are well known to one skilled in the art.

The oocyte and spermatid or sperm head can be from any compatible source, such as from the same species.

In one embodiment, the transgenic embryo is implanted into a surrogate mother and allowed to develop into a transgenic animal or plant. The method of implanting a transgenic embryo into a surrogate mother and allowing it to develop into a transgenic offspring are described herein and known to one of ordinary skill in the art (Nagy, A, et al., (“Manipulation of the Mouse Embryos: A Laboratory Manual (Third Edition),” Cold Spring Harbor Laboratory Press, New York, (2003)).

In a particular embodiment, the present invention is a method for obtaining a transgenic embryo comprising incubating a mixture of a transposable exogenous nucleic acid and a transposase or hyperactive mutant of said transposase or a nucleic acid encoding said transposase for about 30 minutes, wherein said exogenous nucleic acid is flanked by at least one inverted repeat of a nucleic acid sequence comprising SEQ ID NO: 1, SEQ ID NO: 2, or SEQ ID NO: 3 and said transposase is a hyperactive Tn5 transposase mutant comprising an amino acid sequence of SEQ ID NO: 4 or encoded by a nucleic acid sequence comprising SEQ ID NO: 5 or a codon biased nucleic acid sequence comprising SEQ ID NO: 6; contacting said mixture with a sperm head for about 2 minutes; and introducing said mixture contacted with said sperm head into an unfertilized metaphase II oocyte by microinjection to form a transgenic embryo; whereby said transposase catalyzes integration of said transposable exogenous nucleic acid into the genome of said embryo. The present invention can further comprise implanting the transgenic embryo into a surrogate mother and allowing the embryo to develop into a transgenic animal or plant.

Standard techniques for cloning, DNA isolation, amplification (Polymerase Chain Reaction (PCR)), purification, hybridization, and other variously employed techniques, whether or not described in detail herein, are well known to those of ordinary skill in the art. Common references in the art include: Sambrook et al., Eds., “Molecular Cloning: A Laboratory Manual,” 2nd edition, Cold Spring Harbor University Press, New York (1989); and Ausubel et al., Eds., “Current Protocols In Molecular Biology,” John Wiley & Sons, New York (1998).

In another embodiment, the present invention is a method for generating a transgenic embryo comprising incubating a mixture of a transposable exogenous nucleic acid and a transposase or a hyperactive mutant of said transposase or a nucleic acid encoding said transposase; and introducing said mixture into an in vitro fertilized (IVF) oocyte to form a transgenic embryo; whereby said transposase catalyzes integration of said transposable exogenous nucleic acid into the genome of said embryo. Transposase-mediated in vitro fertilization (TN:IVF), as used herein, refers to catalyzing integration of a transposable exogenous nucleic acid into the genome of an in vitro fertilized oocyte with a transposase to form a transgenic embryo. The present invention can further comprise implanting the transgenic embryo into a surrogate mother and allowing it to develop into a transgenic animal or plant. The transposable exogenous nucleic acid and the transposase or a hyperactive mutant of said transposase or a nucleic acid encoding said transposase can be introduced into the oocyte by microinjection, electroporation, liposome vesicles, viral infection, and particle bombardment.

In a particular embodiment, the present invention is a method for generating a transgenic embryo comprising incubating a mixture of a transposable exogenous nucleic acid and a transposase or a hyperactive mutant of said transposase or a nucleic acid encoding said transposase for about 30 minutes, wherein said exogenous nucleic acid is flanked by at least one inverted repeat of a nucleic acid sequence comprising SEQ ID NO: 1, SEQ ID NO: 2, or SEQ ID NO: 3 and said transposase is a hyperactive Tn5 transposase mutant comprising an amino acid sequence of SEQ ID NO: 4 or encoded by a nucleic acid sequence comprising SEQ ID NO: 5 or a codon biased nucleic acid sequence comprising SEQ ID NO: 6; and introducing said mixture into an in vitro fertilized oocyte by microinjection to form a transgenic embryo; whereby said transposase catalyzes integration of said transposable exogenous nucleic acid into the genome of said embryo.

Methods of preparing in vitro fertilized (IVF) oocytes are well known to one of ordinary skill in the art (Rogers B J, et al., Gamete Res 1:165-223 (1978); Nagy, A, et al., “Manipulation of the Mouse Embryos: A Laboratory Manual (Third Edition),” Cold Spring Harbor Laboratory Press, New York, (2003)).

The present invention can also be used to generate transgenic embryos and animals or plants in organisms that do not have sufficient motile sperm but do possess round spermatids. Thus, the present invention can generate transgenic offspring from conventionally sterile males.

In one embodiment, the present invention is a method for obtaining a transgenic embryo comprising incubating a mixture of a transposable exogenous nucleic acid and a transposase or a hyperactive mutant of said transposase or a nucleic acid encoding said transposase; contacting said mixture with a round spermatid; and introducing said mixture contacted with said spermatid into an artificially activated oocyte to form a transgenic embryo, whereby said transposase catalyzes integration of said exogenous nucleic acid into the genome of said embryo. Round spermatid and spermatid, as used herein, refer to immature haploid male gametes.

Methods of preparing artificially activated oocytes and harvesting round spermatids are described herein and are known to one skilled in the art.

In a particular embodiment, the present invention is a method for obtaining a transgenic embryo comprising incubating a mixture of a transposable exogenous nucleic acid and a transposase or a hyperactive mutant of said transposase or a nucleic acid encoding said transposase for about 30 minutes, wherein said exogenous nucleic acid is flanked by at least one inverted repeat of a nucleic acid sequence comprising SEQ ID NO: 1, SEQ ID NO: 2, or SEQ ID NO: 3 and said transposase is a hyperactive Tn5 transposase mutant comprising an amino acid sequence of SEQ ID NO: 4 or encoded by a nucleic acid sequence comprising SEQ ID NO: 5 or a codon biased nucleic acid sequence comprising SEQ ID NO: 6; contacting said mixture with a round spermatid for about 2 minutes; and introducing said mixture contacted with said spermatid into an artificially activated oocyte by microinjection to form a transgenic embryo, whereby said transposase catalyzes integration of said transposable exogenous nucleic acid into the genome of said embryo. The present invention can further comprise implanting the transgenic embryo into a surrogate mother and allowing the embryo to develop into a transgenic animal or plant.

The present invention also encompasses the use of transposases to catalyze the integration of transposable exogenous nucleic acids into the genome of embryonic stem cells (ES). In a particular embodiment, the transposase or a nucleic acid encoding a transposase and the transposable exogenous nucleic acid is introduced into an embryonic stem cell by electroporation, whereby the transposase catalyzes the integration of the exogenous nucleic acid into the genome of the embryonic stem cell.

The present invention is also useful for generating cloned transgenic embroyos and animals. In one embodiment, the present invention is a method to obtain a cloned transgenic embryo comprising removing a nucleus from an oocyte to form an anucleated oocyte; incubating a mixture of a transposable exogenous nucleic acid and a transposase or a hyperactive mutant of said transposase or a nucleic acid encoding said transposase; introducing said mixture and an exogenous diploid nucleus into said anucleated oocyte to form a nucleated oocyte; and activating said nucleated oocyte to form a cloned transgenic embryo; whereby said transposase catalyzes integration of said transposable exogenous nucleic acid into the genone of said cloned embryo to form a cloned transgenic embryo. The method may further comprise implanting the cloned transgenic embryo into a surrogate mother and allowing the embryo to develop into a live cloned transgenic animal.

Methods for cloning animals via nuclear transfer are known in the art (Wakayama T, et al., Nature 394:369-374 (1998)). Activation of oocytes is described herein and known in the art. Cloned transgenic, as used herein, refers to a cloned embryo or animal that contains exogenous nucleic acids. Anucleated oocyte refers to an oocyte that has had its nucleus removed. Exogenous diploid nucleus, as used herein, refers to a diploid nucleus that is external to the cell in which it is introduced. Diploid refers to a cell which has two sets of chromosomes. Nucleated oocyte, as used herein, refers to an oocyte that has undergone nuclear transfer. Cloned embryo, as used herein, refers to an oocyte that has received an exogenous nucleus and develops into an embryo.

The present invention is further described below through examples which are not intended to be limiting.

### EXAMPLES

Applicants have developed novel methods for mouse transgenesis. The invention relies on a hyperactive Tn5 transposase mutant to insert a transgene into mouse chromosomes during intracytoplasmic sperm injection. This procedure integrates foreign DNA into the mouse genome with increased effectiveness as compared to conventional methods such as pronuclear microinjection and traditional intracytoplasmic sperm injection-mediated transgenesis. The data indicate that with this method, transgenic mice, both hybrids and inbreds, can be produced more consistently and with lower numbers of manipulated oocytes than is required for traditional microinjection methods. The transposase-mediated transgenesis technique is also effective with round spermatids, offering the potential for rescuing the fertility of azoospermic animals using sperm precursor cells.

### Background

Gene delivery and production of transgenic animals are becoming increasingly important in every aspect of basic and applied research with many transgenic mice strains serving as important disease models. There are several approaches for producing transgenic animals by the introduction of recombinant DNA into their somatic or germ cells (Nakanishi T et al., Genomics 80: 564-574 (2002); Perry A C et al., Science 284: 1180-1183 (1999); Lois C et al., Science 295: 868-872 (2002); Wall R J Theriogenology 57: 189-201 (2002)). To achieve ubiquitous cellular expression of recombinant DNA, most transgenesis efforts have concentrated on the insertion of a transgene (tg) at the unicellular stage of developing embryos. The effective insertions of such transgenes have been achieved primarily by passive means, such as pronuclear microinjection (Nakanishi T et al., Genomics 80: 564-574 (2002)) and traditional intracytoplasmic sperm injection-mediated transgenesis (ICSI-Tr) (Perry A C et al., Science 284: 1180-1183 (1999)). However, the most effective means of tg insertion to date is exemplified by the active Lentiviral transgenesis technique which makes use of viral sequences and enzymes to increase the efficiency of tg insertion (Lois C et al., Science 295: 868-872 (2002)).

Passive transgenesis entails the injection of linearized tg DNA into the pronuclei of single celled embryos, or it's co-microinjection with freeze-thawed spermatozoa into the cytoplasm of mature unfertilized oocytes by ICSI-Tr (Perry A C et al., Science 284: 1180-1183 (1999); U.S. Pat. No. 6,376,743). Transgenes in the vicinity or within the nuclei of an individual in its very initial stages of development rely for insertion on the DNA repair mechanisms of their new-found environment (Yanagimachi R “Mammalian Fertilization” in Knobil E, Neill, J D (Ed.) “The Physiology of Reproduction,” 2nd Edition. New York: Raven Press, (1994); Perry A C, Mol Reprod Dev 56: 319-324 (2000)). When insertion does occur, it is about 1 % to 5% of oocytes micromanipulated (oi) (Nakanishi T, et al., Genomics 80: 564-574 (2002); Perry A C, et al., Science 284: 1180-1183 (1999); Moreira P N, et al., Biol Reprod 71: 1943-1947 (2004); Wall R J, Cloning Stem Cells 3: 209-220 (2001); Hirabayashi M, et al., Exp Anim 50: 125-131 (2001); Perry A C, et al., Nat Biotechnol 19: 1071-1073 (2001)). In the case of ICSI-Tr, such micromanipulations as freeze-thawing spermatozoa have a detrimental effect on the development of early embryos (Szczygiel M A, et al., Biol Reprod 68: 1903-1910 (2003)). It is also common that tg's integrated by passive transgenesis commonly form long concatemeric arrays. Such transgene loci are not desirable due to their potential to generate aberrant RNAs that can cause gene silencing (Garrick D, et al., Nat Genet 18: 56-59 (1998)).

The active Lentiviral technique uses disarmed retroviral vectors to insert desirable genes into the host organism, usually single celled embryos (Lois C, et al., Science 295: 868-872 (2002); Hofmann A, et al., EMBO Rep 4: 1054-1060 (2003)). However, there are drawbacks to this technique as exemplified by high embryo lethality rates (70-80% of oi) and the relatively small size of tg DNA (9.5 kilobase pairs (kb)) that can be transported by the vector (Lois C, et al., Science 295: 868-872 (2002); Whitelaw C B, Trends Biotechnol 22: 157-160 (2004)). Such limitations, in combination with the requirement of specialized containment facilities for retroviral production, inhibit most laboratories from using the active retroviral transgenesis approach. Despite its potential safety problems, a high percentage (˜80%) of the founder (F0) animals born (ab) after Lentiviral transgenesis carry several (1-20) stably inserted tg copies (Lois C, et al., Science 295: 868-872 (2002)). This high efficiency of transgenic F0 production (23% of oi) has attracted considerable interest as a new transgenesis method in the livestock industry where the cost of producing one transgenic cow by pronuclear microinjection is estimated to be about $300,000 (Whitelaw C B, Trends Biotechnol 22: 157-160 (2004); Hofmann A, et al., Biol Reprod 71: 405-409 (2004)).

The development of alternative active transgenesis methods that are more flexible in regards to tg size and less problematic than the Lentiviral system in terms of biosafety considerations, while being more efficient than conventional ICSI-Tr, would be advantageous. Applicants utilized a Tn5 transposase mutant. The Tn5 transposase is a well-characterized bacterial transposase for transposon delivery. The structure and mechanism for Tn5 transposase are established for both wild type and mutants (Reznikoff W S, Mol Microbiol 47: 1199-1206 (2003); Peterson G and Reznikoff W, J Biol Chem 278: 1904-1909 (2003); Goryshin I Y and Reznikoff W S, J Biol Chem 273: 7367-7374 (1998); Naumann T A and Reznikoff W S, J Biol Chem 277: 17623-17629 (2002); Naumann T A and Reznikoff W S, J Bacteriol 184: 233-240 (2002)). Unlike the wild type transposases, the hyperactive Tn5 mutant transposase, designated herein as *Tn5p, containing three amino acid mutations (E54K, M56A, L372P), exhibits a high transposon insertion activity in vitro and has been used to produce DNA:transposase complexes with the transposase protein bound to both ends of transposon DNA (Reznikoff W S, Mol Microbiol 47: 1199-1206 (2003); Goryshin I Y and Reznikoff W S, J Biol Chem 273: 7367-7374 (1998); Naumann T A and Reznikoff W S, J Biol Chem 277: 17623-17629 (2002)). Such complexes, called transposomes, are formed by the transposase protein binding to specific 19 bp recognition Mosaic End (ME) sequences of the transposon in the absence of magnesium ions (Mg2+). Transposomes have subsequently been used for crystallization studies (Davies D R, et al., Science 289: 77-85 (2000)) as well as for bacterial gene delivery (Goryshin I Y and Reznikoff W S, J Biol Chem 273: 7367-7374 (1998)) where, following electroporation, the transposase becomes activated by cellular Mg2+ levels and integrates the transposon DNA into a random position favoring GATC(A/T)GATC sequences in bacterial chromosomes (Goryshin I Y, et al., Proc Natl Acad Sci USA 95: 10716-10721 (1998); Goryshin I Y, et al., Genome Res 13: 644-653 (2003)).

Applicants report the production of hybrid and inbred transgenic mice, using a modified transposome-assisted microinjection technique. In vitro synthesized transposomes were injected into mouse oocytes together with fresh sperm heads (TN:ICSI) or round spermatids (TN:ROSI). Activation of the transposome complex by the Mg²⁺-rich oocyte cytoplasm results in transgenesis with transgenic animals born, thus demonstrating enhanced green fluorescent protein (EGFP) tg expression in their tissues under ultraviolet light illumination. This technique allows for the use of unfrozen sperm in transgenesis, resulting in a significantly higher percentage of live births and a larger proportion of transgenic animals, while using fewer microinjected oocytes.

### Materials and Methods

Animals

Females and males of B6D2F1 (B57BL/6×DBA/2), C57BL/6, and CD1 mice were purchased from the National Cancer Institute (Raleigh, N.C.). All animals were maintained in temperature- and light-controlled rooms (14 hours light/10 hours dark; light on from 5:00 a.m.). The protocol of animal handling and treatment was reviewed and approved by the Animal Care and Use Committee of the University of Hawaii.

Construction of Transposon DNA

The plasmid pCX-EGFP, expressing EGFP under the control of (operably linked to) the CAG promoter, was a kind gift from Dr. Masaru Okabe (Ikawa M, et al., FEBS Lett 375: 125-128 (1995)). CAG is a composite promoter that combines the human cytomegalovirus immediate-early enhancer and a modified chicken beta-actin promoter and first intron (Niwa H, et al., Gene 108:193-9 (1991)). Other suitable promoters could be used and are well known to one skilled in the art. The 3179 bp SalI/BamHI restriction enzyme-cleaved DNA fragment containing the EGFP gene and its regulatory elements was cloned into SalI/BamHI sites present in the multiple cloning site (MCS) of the plasmid pMOD-3<Rγori/MCS> (Epicentre, Madison, Wis.). The transposon, flanked by its ME sequences (CTGTCTCTTATACACATCT (SEQ ID NO: 1)), was excised from the resulting plasmid pMOD-3/CX-EGFP by digestion with the restriction endonuclease PshAI (New England Biolabs, Inc., Beverly, Mass.), and the 3608 bp DNA fragment containing the active transposon was gel-purified using standard methods and then used for transposome assembly.

Preparation of Transposome Complex

A 6 microliters (μl) reaction solution was prepared by mixing together 2 μl of 100 nanograms per microliter (ng/μl) EZ:TN transposon DNA (Epicentre, Madison, Wis.) containing the pCX-EGFP gene in TE buffer (10 mM Tris-HCl (pH 7.5), 1 mM EDTA) and 4 μl EZ:TN transposase (1U/μl) (Epicentre, Madison, Wis.). After mixing the transposon and the transposase, the reaction was incubated at room temperature for 30 minutes (min) to allow the formation of the transposome.

Preparation of Microinjection Media

CZB medium supplemented with 5.56 mM D-glucose, referred to herein as CZB, was used for the culture of mouse oocytes after microinjection (Chatot, C L et al., J. Reprod. Fertil. 86:679-688 (1989)). The medium for oocyte collection and subsequent oocyte treatments, including micromanipulation, was a HEPES-modified CZB medium (referred to as HEPES-CZB medium; CZB modified to contain 20 mM HEPES-HCl, 5 mM NaHCO3, pH 7.4, and 0.1 mg/ml polyvinyl alcohol (PVA; cold water soluble; Mr 30,000-70,000) instead of bovine serum albumin (Kimura Y and Yanagimachi R, Biol Reprod 52: 709-720 (1995)). CZB medium was used under 5% CO₂ in air and HEPES-CZB was used under air. The medium for microinjection was Mg²⁺-free HEPES-CZB.

ICSI Microinjection

ICSI was carried out essentially as described by Kimura and Yanagimachi using a Piezo electric micropipette actuator (Kimura Y and Yanagimachi R, Biol Reprod 52: 709-720 (1995)), except that the manipulation was carried out at room temperature (about 25° C). Briefly, epididymal spermatozoa and matured oocytes were collected from 8- to 12-week-old B6D2F1 hybrid (progeny of B57BL/6 ×DBA/2; F1 means first filial generation) or C57BL/6 inbred mice. Recipients of 2-cell embryos were 8- to 16-week-old out-bred CD-1 females. Oocytes were collected from oviducts of superovulated B6D2F1 or C57BL/6 females after intraperitoneal injection of 5 international units (IU) pregnant mare serum gonadotropin (PMSG) followed by injection of 5 IU human chorionic gonadotrophin (hCG) 48 hours later. Matured oocytes collected 13-15 hours (h) after hCG injection were freed from cumulus cells by treatment with 0.1% bovine testicular hyaluronidase (359 units/miligram (mg) solid) in HEPES-CZB medium. The oocytes were rinsed and kept at 37° C. in fresh HEPES-CZB medium before sperm injection. Spermatozoa were collected from the cauda epididymis of B6D2F1 or C57BL/6 males. A dense sperm mass that was squeezed out of the epididymis was placed at the bottom of 200 μl Mg²⁺ free HEPES-CZB buffered solution in a microcentrifuge tube. After standing for 10 min at 37° C. the upper 20 μl of the sperm suspension was collected and mixed with an equal volume of 12% polyvinylpyrollidone (PVP) solution. A single spermatozoon moving slowly in the solution was drawn, tail first, into the injection pipette in such a way that its neck (the junction between the head and tail) was at the opening of the pipette. The head was separated from the tail by applying a few Piezo-pulses to the neck region. The sperm head was transferred to a 20 μl Mg²⁺ free HEPES-CZB containing 12% PVP and an appropriate concentration of transposome mixture (approximately 16.2 ng/μl transposon DNA). One minute later, sperm heads were individually injected into oocytes. ICSI-oocytes were cultured in CZB medium at 37° C. under 5% CO₂ in air.

ROSI Microinjection

Mouse round spermatids are the smallest cells in the testis and are characterized by a centrally located chromatin mass (Ogura A, et al., Proc Natl Acad Sci USA 91: 7460-7462 (1994); Kimura Y and Yanagimachi R, Development 121: 2397-2405 (1995)). Round spermatids were collected from the testes of B6D2F1 males and microinjected into oocytes according to Kimura and Yanagimachi (Development 121: 2397-2405 (1995)), with some modifications. Briefly, round spermatids were placed in Mg²⁺-free Hepes-CZB medium containing 12% PVP. The round spermatids were then transferred to a 20 μl Mg2+ free HEPES-CZB solution containing 12% PVP and an appropriate amount of transposome mixture, resulting in the final concentration of 16.2 ng/μl transposon DNA. After mixing for 1 minute, several round spermatids were drawn into a micropipette. The plasma membrane of each spermatid was broken by sucking it in and out of the pipette and nuclei were individually injected into mice oocytes, which had been previously activated by a 30 minute treatment with 10 mM SrCl₂ in a Ca²+-free CZB medium (Shamanski F L, et al., Hum Reprod 14: 1050-1056 (1999); Kline D and Kline J T, Dev Biol 149: 80-89 (1992)). ROSI oocytes were cultured in CZB medium under 5% CO₂.

Pronuclear and Cytoplasmic Microinjections

Transposomes or corresponding amounts of double stranded DNA (dsDNA) were injected directly into cytoplasm or male pronuclei of B6D2F2 (second generation; F1×F1) zygotes using an InjectMan microinjection apparatus (Eppendorf, Westbury, N.Y.).

Embryo Culture and Embryo Transfer

ICSI or ROSI oocytes with two well developed pronuclei and a distinct second polar body 5 to 6 h after injection of spermatozoa or round spermatids were recorded as being normally fertilized. They were cultured in CZB medium under 5% CO₂ until they reached the 2-cell stage (20-24 h after microinjection). They were then transferred into the oviducts of 8- to 16-weeks-old surrogate pseudopregnant CD-1 females which were mated with vasectomized males of the same strain on the day before embryo transfer (Kimura Y and Yanagimachi R, Biol Reprod 52: 709-720 (1995); Kimura Y and Yanagimachi R, Development 121: 2397-2405 (1995); Kimura Y and Yanagimachi R, Biol Reprod 53: 855-862 (1995)). Pregnant females were allowed to deliver and raise their pups.

Analysis of Offstring

Genomic DNA obtained from tail-tip biopsies of EGFP-negative 30-day-old offspring was analyzed by polymerase chain reaction (PCR) for the presence of transgene sequences. Forward primer (atggtgagcaagggcgaggagctgttcacc, position 0 to 30) (SEQ ID NO: 7) for 5′- end of EGFP and reverse 28 bp primer (cttgatgccgttcttctgcttgtcggcc, position 490 to 462) (SEQ ID NO: 8) for middle part of EGFP were used to amplify a 490 bp EGFP fragment. Reaction parameters were: 94° C. for 3 minutes (min) (1 cycle); 94° C. for 30 seconds (s), 58° C. for 30 s, 72° C. for 30 s (35 cycles), with a final extension at 72° C. for 3 min. For Southern blot analyses, 20 μg genomic DNA per sample was cleaved with the restriction enzyme NcoI and separated by gel electrophoresis in 1% agarose gels. The DNA was transferred to Immobilon nylon-membranes and probed with a 416 base pairs (bp) digoxigenin (DIG)-labeled DNA probe corresponding to the 3′-part of the EGFP gene and its rabbit beta-globin polyA signal. Probe hybridization was detected using the DIG-labeling and hybridization kit (Roche, Alameda, Calif.). Precise insertion of full-length transposons into the genome of the 23 Southern blot-detected TN:ICSI EGFP positive mice was analyzed by PCR with primers designed to the 5′-end and 3′-end regions of the 3608 bp long transposon. The forward primer for the 5′- end of the transposon (ctgtctcttatacacatctcaaccatcatcg, position 1 to 31) (SEQ ID NO: 9), and the 22 bp reverse primer (cctgactactcccagtcatagc, positions 339 to 317) (SEQ ID NO: 10) were used to amplify the 5′-end region. The 3′- end forward primer (gtgaaacatgagagcttagtacg, position 3397 to 3419) (SEQ ID NO: 11) and the corresponding reverse primer (ctgtctcttatacacatctcaaccctgaagc, position 3608 to 3578) (SEQ ID NO: 12) were used to amplify the 3′-end region. Conditions for the PCR reactions were: 94° C. for 3 min (1 cycle); 94° C. for 30 s, 58° C. for 30 s, 72° C. for 30 s (40 cycles); with a final extension at 72° C. for 3 min.

Results

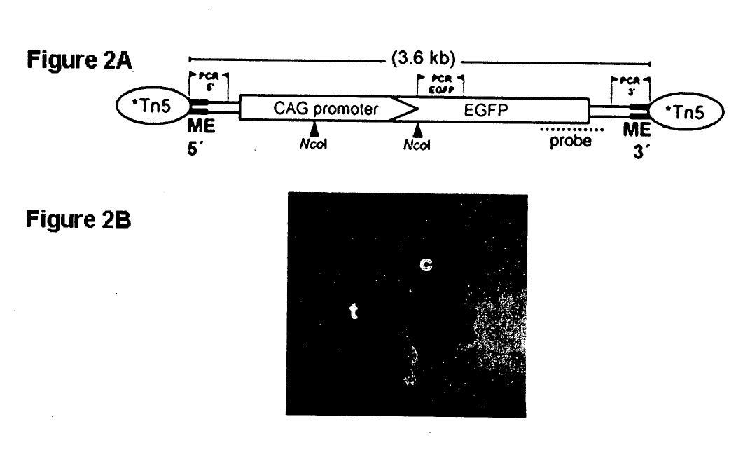

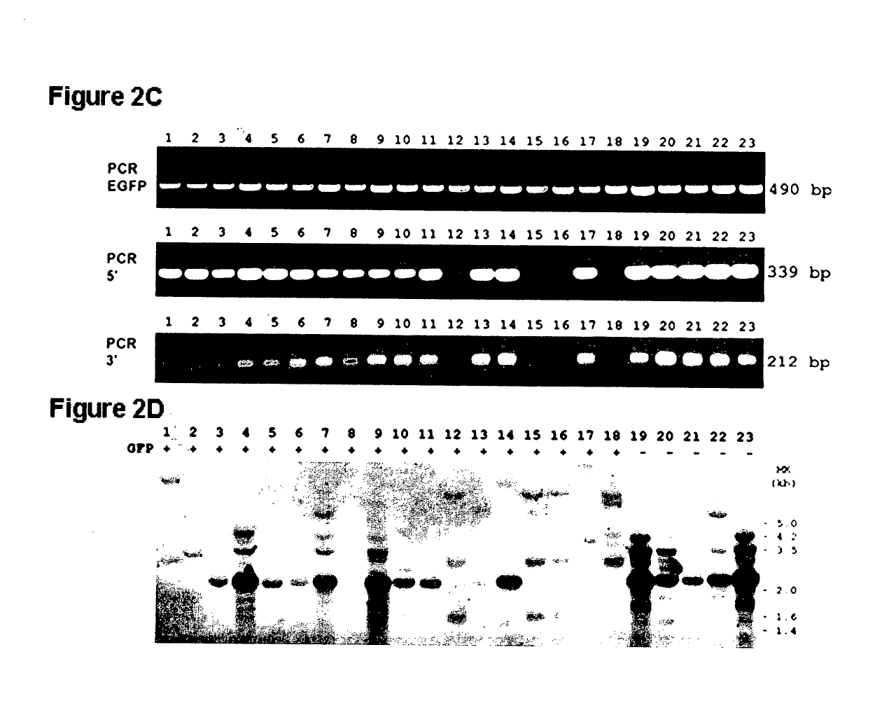

In this study, Applicants employed ICSI as well as other microinjection-based methods for transgenesis of hybrid (B6D2F1) and inbred (C57BL/6) strains of mice. The delivery and integration of EGFP-coding tg's into the mouse embryo genome was carried out with the help of a hyperactive mutant of the Tn5 transposase protein designated *Tn5p (Reznikoff W S, Mol Microbiol 47: 1199-1206 (2003); Naumann T A and Reznikoff W S, J Biol Chem 277:17623-17629 (2002)) (FIG. 1A). The *Tn5p:DNA complexes or “transposomes” resembling natural Tn5 transposition intermediates were formed by allowing the purified transposase to bind to its ME recognition sequences (SEQ ID NO: 1) in the absence of Mg²⁺ ions (FIG. 1A). Freshly isolated sperm heads were individually co-injected into mouse metaphase II (MII) oocytes with either naked dsDNA alone, as described in previous ICSI transgenesis studies (ICSI-Tr) (Perry A C, et al., Science 284: 1180-1183 (1999); Perry A C, et al., Nat Biotechnol 19: 1071-1073 (2001)), or as a *Tn5p:DNA complex (TN:ICSI) (FIG. 1B). The DNA fragment used to construct the transposome contained an EGFP gene driven by a CAG promoter (Ikawa M, et al., FEBS Lett 375: 125-128 (1995)) (FIG. 2A). Two-cell embryos were then transferred into oviducts of surrogate females and allowed to develop to term (FIG. 1B). All of the resulting F0 transgenic progeny (t) were recognized for tg expression by epifluorescence of EGFP⁺ (FIG. 2B). Control progeny (c) do not exhibit epifluorescence. In a control experiment (ICSI using only transposon DNA), only one pup was germline transgenic and showed weak mosaicism (data not shown).

TABLE 1
Summary of TN:ICSI, TN:ROSI, pronuclear and cytoplasmic injection experiments

| | | | | Number of | | | | |
|---|---|---|---|---|---|---|---|---|
| | | Number of | Total number | embryos | | | Transgenic | |
| | Strain of mice | oocytes | of normally | transferred | Births | | Pups | |
| Transgensis | used for sperm | injected | fertilized | (surrogate | (% transferred) | | (% births): | (% oocytes) |
| method | and oocyte | (repetitions) | oocytes | mothers) | [% oocytes] | Total: | ab | oi |
| (A) | | | | | | | | |
| Transposome ICSI | B6D2F1 Hybrid | 204 (7) | 182 | 171 (14) | 107 | 23 | 21.5 | 11.3 |
| | | | | | (62.6) | | | |
| | | | | | [52.0] | | | |
| Frozen sperm | B6D2F1 Hybrid | 50 (1) | 45 | 39 (3) | 20 | 1 | 5.0 | 2.0 |
| Transposome ICSI | | | | | (51.0) | | | |
| | | | | | [40.0] | | | |
| Transposome ICSI | C57BL/6 Inbred | 94 (2) | 84 | 77 (6) | 45 | 4 | 8.8 | 4.3 |
| | | | | | (58.4) | | | |
| | | | | | [47.9] | | | |
| DNA-only ICSI | B6D2F1 Hybrid | 106 (2) | 99 | 87 (6) | 40 | 1 @ | 2.5 | 0.9 |
| Control | | | | | (46.0) | | | |
| | | | | | [37.5] | | | |
| (B) | | | | | | | | |
| Transposome ROSI | B6D2F1 Hybrid | 120 (5) | 108 | 86 (7) | 31 | 5 | 16.1 | 4.2 |
| (#) | | | | | (36.0) | | | |
| | | | | | [25.8] | | | |
| DNA-only ROSI | B6D2F1 Hybrid | 49 (1) | 44 | 33 (2) | 11 | 0 | 0 | 0 |
| control (#) | | | | | (33.3) | | | |
| | | | | | [22.5] | | | |
| (C) | | | | | | | | |
| Transposome | B6D2F2 Hybrid | 160 (4) | 117 | 105 (6) | 47 | 1 | 2.1 | 0.63 |
| Pronuclear | | | | | (45) | | | |
| microinjection | | | | | [30] | | | |
| DNA-only | B6D2F2 Hybrid | 40 (1) | 31 | 26 (2) | 10 | 0 | 0 | 0 |
| Pronuclear micro | | | | | (38.5) | | | |
| injection control | | | | | [25] | | | |
| (D) | | | | | | | | |
| Transposome | B6D2F2 Hybrid | 136 (4) | 119 | 115 (8) | 79 | 1 | 1.3 | 0.74 |
| Cytoplasmic | | | | | (68.7) | | | |
| injection | | | | | [58] | | | |
| DNA-only | B6D2F2 Hybrid | 59 (1) | 54 | 42 (4) | 18 | 0 | 0 | 0 |
| Cytoplasmic | | | | | (42.9) | | | |
| injection control (§) | | | | | [31] | | | |

@ = Very weak chimera: germline transgenic
(#) = Activation of oocytes 30 minutes before ROSI microinjection
(§) = Embryos at two pronuclei stage

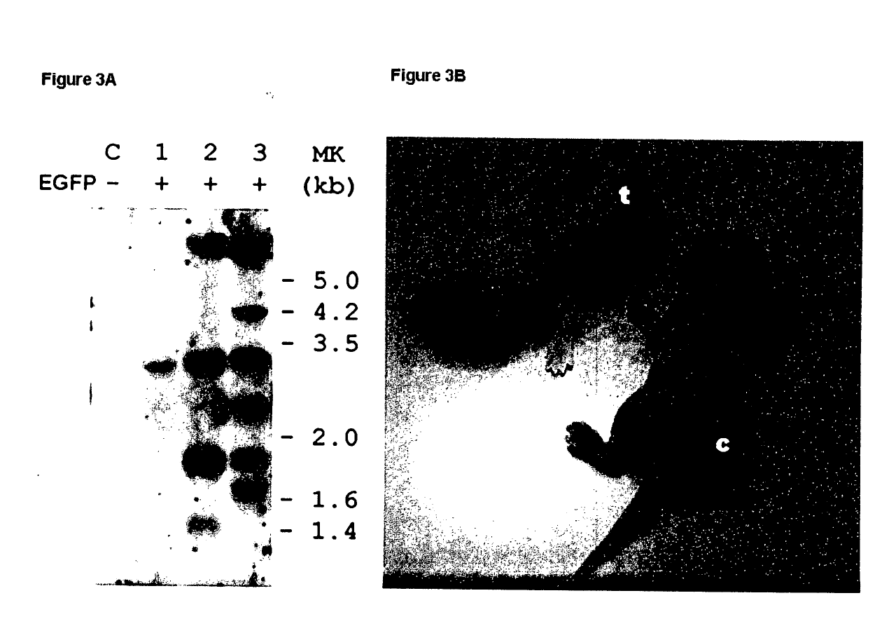

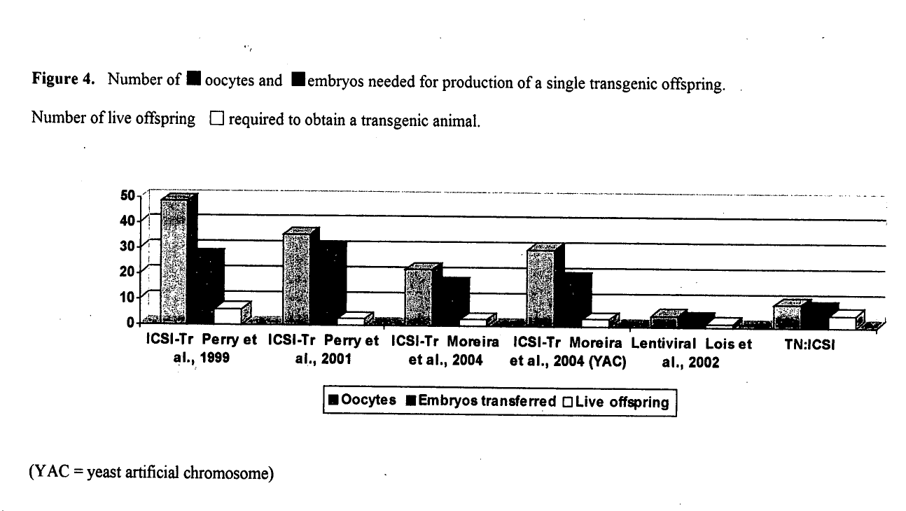

The data in Table 1 is a summary of all micromanipulations employed in this study. Panel A represents the combined data from seven ICSI microinjection repetitions with approximately an average of 29 oocytes per repetition and attempts with two inbred mouse strains with an average of 47 oocytes per repetition. All such TN:ICSI attempts result in the production of live transgenic pups, including a microinjection attempt where only 14 oocytes where subjected to TN:ICSI; this hybrid strain attempt with low oocyte numbers produced a single transgenic live born pup (FIG. 2B). Panel B exhibits ROSI microinjections generated data, with an average of 24 oocytes per microinjection attempt. Each attempt resulted in a live born transgenic pup, giving a total of five such animals (FIG. 3B). Panel C depicts pronuclear microinjection attempts and Panel D contains cytoplasmic microinjection data.

PCR and Southern Blotting

All live born pups were screened by PCR for EGFP tg integration with primers indicated in FIG. 2A. The ones found to be positive for the tg were further challenged for full length tg insertion (FIG. 2C) and their genomic DNA was subjected to Southern blotting to identify tg copy number (FIG. 2D). From these, 23 PCR-positive for EGFP, F0 B6D2F1 hybrid animals were confirmed for tg integration (FIGS. 2C and 2D). Fragments corresponding to perfectly preserved 5′ and 3′ ends of the transposome were detected in 22/23 animals, indicating a high degree of transgene preservation prior to integration, probably due to protection of DNA ends by bound transposase molecules (FIG. 2C). The number of tg's ranged from 1 to ˜20 with 6 out of 23 animals carrying just 1 or 2 copies of the tg (FIG. 2D, lanes 1, 2, 3, 8, 13 and 17). Lanes 4, 7, 9, 14, 19, 20, 22 and 23 of FIG. 2D additionally contain a strong band in the region of 2.4 kb that resembles concatemerized fragments produced from head to tail integration. Three lanes (12, 15, and 16, FIG. 2D) depict insertions demonstrating a similar pattern and suggest the possible existence of common insertion sites for *Tn5p in the mouse genome. Insertion site analyses with rescue plasmids will elucidate this question and lead to a better understanding of the transposition reactions for *Tn5p in mammals. Such insertion site preferences for target DNA have been demonstrated in bacterial transposition experiments (Goryshin I Y, et al., Proc Natl Acad Sci USA 95: 10716-10721 (1998)).

Meiotic Transmission of Transgene

Analysis of F1 progeny from crosses between EGFP expressing F0 hybrid founders and non-transgenic partners established that germline transmission of the tg was approximately 3 to 1, indicating single or closely linked integration sites (Table 2). Southern blots of genomic DNA obtained from biopsies of F 1 progeny mirrored the tg insertion patterns of the parents (data not shown).

TABLE 2
Meiotic transmission of EGFP gene expression in
F₁ offspring, from F₀ (+EGFP) × wt cross

| Transgensis method | Sex of F | Number of EGFP + | % EGFP |
|---|---|---|---|
| (# animal I.D.) | mice 0 | pups in F litter 1 | positive |
| Fresh sperm transposome | | | |
| ICSI | | | |
| #1 | F | 2/6 | 33.3 |
| #2 | M | 9/19 | 47.4 |
| #3 | F | 4/10 | 40.0 |
| #4 | F | 2/6 | 33.3 |
| #5 | M | 5/9 | 55.6 |
| #6 | M | 4/6 | 66.7 |
| #7 | M | 6/19 | 31.6 |
| #8 | F | 3/7 | 42.9 |
| #9 | F | 3/9 | 33.3 |
| #10 | M | 6/17 | 35.3 |
| #11 | F | 5/9 | 55.6 |
| #12 | F | 2/6 | 33.3 |
| #13 | F | 1/7 | 14.3 |
| #14 | F | 2/9 | 22.2 |
| #15 | M | 4/10 | 40.0 |
| #16 | M | 10/18 | 55.6 |
| #17 | M | 7/10 | 70.0 |
| #18 | F | 3/8 | 37.5 |
| Control transposon ICSI | | | |
| #24 | F | 2/5 | 40.0 |
| Freeze-thawed sperm and | | | |
| Transposome ICSI | | | |
| #25 | F | 3/8 | 37.5 |
| Transposome ROSI | | | |
| #R1 | F | 5/9 | 55.6 |
| #R2 | M | 2/7 | 28.6 |

Alternative microinjections

Transgenesis success with TN:ICSI encouraged the Applicants to try *Tn5p-mediated transgenesis by ROSI. Round spermatids, the smallest cells in the testis, were easily recognized by their small size and centrally located chromatin mass. Transposomes were co-injected with a round spermatid into the cytoplasm of an artificially activated mature unfertilized oocyte. This new ROSI based transgenesis approach (TN:ROSI) resulted in 5 transgenic EGFP-expressing pups (Table 1, Panel B) corresponding to transgenesis efficiencies of 4.2% oi and 16.1% ab. Southern analysis done on the first three born F0 TN:ROSI animals revealed a presence of 1, 7 and 10 copies of the tg, respectively (FIG. 3A). Similar to the case of TN:ICSI generated transgenics, the segregation of the EGFP expression for 2 F0 ROSI mice in the F1 generation suggested a single locus integration of the tg (Table 2, # R1 and R2). Southern analysis of genomic DNA blots obtained from biopsies of TN:ROSI F1 progeny mirrored the transgene insertion patterns of the parents (data not shown).

Applicants injected transposomes into the pronuclei or the cytoplasm of single-cell embryos of B6D2F 1 hybrid mice (Table 1, Panels C and D). Somewhat surprisingly, neither pronuclear nor cytoplasmic injection of transposomes into single celled embryos resulted in efficient transgenesis (Table 1, Panels C and D).

Discussion

Applicants have developed a new method of active transgenesis that is more flexible than the Lentiviral system in terms of tg size, and less problematic in terms of biosafety considerations. Although retroviral vectors have a gene delivery approaching 80% transgenesis efficiencies with respect to ab (Lois C, et al., Science 295: 868-872 (2002))—most transgenic animals, especially mice, are produced by classical, less efficient, pronuclear microinjection methods due to the drawbacks listed previously.

Scoring transgenesis efficiency is a contentious matter. In techniques where post embryo transfer development is poor, the preferred way is to score the efficiency as a ratio of animals-born/animals-transgenic (ab/at). Under such conditions, 6 transgenic animals in a litter of 14 live born represents an efficiency of 43%, irrespective of the number of embryos transferred into the surrogate mothers (179 in the case of this example) (Perry A C, et al., Nat Biotechnol 19: 1071-1073 (2001)). However, an investment had to be made to use 12 surrogate mothers and maintain them for the duration of their pregnancy for the many embryos transferred. The choice of transgenesis method is a consideration when utilizing larger animals due to the cost of maintaining these animals. In the case of TN:ICSI, which does not require freeze-thawed sperm for its implementation, 171 two-cell embryos were transferred into 14 surrogate mothers. The result was 107 live born pups of which 23 were transgenic (Table 1, Panel A, FIGS. 2C and 2D), with an efficiency of 21.5% for the ab/at ratio. This improved post embryo transfer development, resulting in so many live births (63%), obliges a researcher to decide whether the researcher would prefer 6 transgenic animals out of 179 embryos transferred with a 43% ab/at ratio, or 23 such animals out of 171 embryos with a 21.5% ab/at ratio. To clarify some of these considerations, Applicants have provided Table 3, which lists the success rates of some recent mouse transgenesis attempts, and FIG. 4, which is a histogram demonstrating the efficiency rates of the different ICSI and Lentiviral microinjection methods to produce a single transgenic offspring. Applicants' TN:ICSI method is the most efficient microinjection method in terms of oocytes used. Surprisingly, the more technically challenging TN:ROSI technique and the TN:ICSI procedure with inbred mice are comparable if not better than some of the other transgenesis techniques (Table 3).

TABLE 3
Comparison of the efficiency of different transgenesis techniques: ICSI-Tr; Lentiviral; Pronuclear microinjection; TN:ICSI; TN:ROSI

| | | | | | | | | Number of | | Efficiency | | |
|---|---|---|---|---|---|---|---|---|---|---|---|---|
| | Method | Male germ | Number of | Number of | Number of | | | transferred | Live | | Germine: | % |
| | used | cell | oocytes used | surviving | embryos reach | | | embryos | offspring | Trans- | Trans- | oo- |
| Authors | (strain) | treatment | (Repetitions) | oocytes | 2 + C^{#} | m | b | (Recipients) | (% oocytes) | genic: | genic | cytes |
| Perry et | ICSI | Freeze- | 97 (1) | — | — | 53 | — | 53 (3) | 12 (12.4) | 2 | 2 | 2.0 |
| al., 1999 | (Hybrid) | thaw | | | | | | | | | | |
| Perry et | ICSI | Freeze- | — | (213) | 45 | 164 | 4 | 179 (12) | 14 (6.6)^{a} | 6 | 5 | (2.8)^{a} |
| al., 2001 | (Hybrid) | thaw | | | | | | | | | | |
| Moreira | ICSI | Freeze- | 219 (6) | 195 | 152 | — | — | 163^{b} (8) | 22 (10.0) | 10 | — | 4.6 |
| et al., 2004 | (Outbred) | thaw | | | | | | | | | | |
| Moreira | ICSI | Freeze- | 367 (6) | 252 | 201 | — | — | 218^{c} (13) | 34 (9.2) | 12 | — | 3.3 |
| et al., 2004 | YAC | thaw | | | | | | | | | | |
| | (Outbred) | | | | | | | | | | | |
| Lois et | Lentiviral | — | 270 (2) | 231 | — | — | — | 197 (—) | 73 (27.0) | 63 | — | 23.3 |
| al., 2002 | (Hybrid) | | | | | | | | | | | |
| Nakanishi | Pronuclear | — | 4739 (—) | — | — | — | — | — (182) | 626 (13.2) | 150 | — | 3.2 |
| et al., 2002 | (Hybrid) | | | | | | | | | | | |
| Present | TN:ICSI | Fresh | 204 (7) | 182 | 171 | — | — | 171 (14) | 107 (52.5) | 23 | 18 | 11.3 |
| invention | (Hybrid) | | | | | | | | | | | |
| | TN:ICSI | Fresh | 94 (2) | 84 | 77 | — | — | 77 (6) | 45 (47.9) | 4 | 4 | 4.3 |
| | (Inbred) | | | | | | | | | | | |
| | TN:ROSI | Fresh | 120 (5) | 108 | 86 | — | — | 86 (7) | 31 (25.8) | 5 | 5 | 4.2 |
| | (Hybrid) | | | | | | | | | | | |

^{a}Calculated from number of surviving oocytes
^{b}11 surviving 1-cell embryos were also transferred
^{c}17 surviving 1-cell embryos were also transferred
— Data not available
2 + C^{#} Embryos at 2 to 8 cell stage
m Morula
b Blastocyst

Fertilization rates obtained using TN:ICSI were comparable to those obtained by ICSI performed with fresh sperm alone (Kimura Y and Yanagimachi R, Biol Reprod 52: 709-720 (1995)) (Tables 1 and 3), indicating that neither naked DNA nor *Tn5p:DNA was deleterious to embryo survival at the concentrations used. TN:ICSI is less technically demanding than TN:ROSI and would be well suited to routine transgenesis. It is efficient in both hybrid and inbred mouse strains and offers the potential of delivering large, gene-sized DNA fragments. The ability to generate transgenic animals with a limited number of oocytes makes it especially well suited for transgenesis attempts on large mammals, such as non-human primates, due to cost considerations. Low copy number, full length, tg integration patterns similar to those obtained by TN:ICSI are preferable in transgenesis experiments, as expression of multiple tg copies or production of “aberrant” RNAs from truncated genes can frequently lead to gene silencing (Garrick D, et al., Nat Genet 18: 56-59 (1998)). This notion is also borne out as described herein, where all five B6D2F2 transgenic animals obtained by TN:ICSI carrying a relatively high number of tg's did not express EGFP (FIG. 2D).

TN:ROSI offers unique possibilities for transgenesis and genetic rescue of azoospermic animals that do not produce the spermatozoa needed for normal sexual or ICSI-mediated fertilization. To date, the only published study describing “pre-sperm” transgenesis was performed with freeze-thawed elongating rat spermatids. The transgenesis success rate with this attempt was a disappointing 0.985% oi (Kato M, et al., Mol Reprod Dev 69: 153-158 (2004)).

Using inbred strains of mice to generate transgenic animals for biomedical research can minimize the problem of genetic variation between individuals. However, the efficiency of transgenesis by pronuclear injection in a strain such as C57BL/6 is only one-eighth of that obtained using a hybrid strain (Brinster R L, et al. Proc Natl Acad Sci USA 82: 4438-4442 (1985)). TN:ICSI transgenesis attempts with the same strain of inbred mice result in an efficiency which is 38% of the TN:ICSI transgenesis with hybrid strains (Table 1, Panel A and Table 3). Therefore the *Tn5p transgenesis method contributes to an increased effectiveness with respect to inbred mouse strain transgenesis.

The procedures described herein can be further optimized in terms of transposome quantity, incubation conditions, or tg size. Such improvements in conditions employed might permit the transposome injection technique to become more effective. Presently, Applicants do not have sufficient information to explain the low transgenesis rates with pronuclei stage embryos, but it is possibly the chromatin decondensation and remodeling that a sperm or a round spermatid nucleus undergoes after injection into a mature oocyte (Kimura Y and Yanagimachi R, Biol Reprod 52: 709-720 (1995); Ramalho-Santos J, et al., Hum Reprod 15: 2610-2620 (2000); Gao S, et al., Dev Biol 266: 62-75 (2004); Chapman J C, et al., Reprod Biol Endocrinol 1: 20 (2003)), may allow an opportunity for transposase enzymes to integrate the transposon into the embryo's genome. Applicants realize that the control DNA-only injections for pronuclear microinjection and cytoplasmic injections are low in number (Table 1, Panels C and D). Successful pronuclear microinjections are achieved routinely elsewhere (Nakanishi T, et al., Genomics 80: 564-574 (2002)). Instead, Applicants' intent was to have transposon controls to compare with transposome injections into the pronucleus and cytoplasm. As these later microinjection techniques are not efficient with transposome injections, the need to produce a higher number of control animals is obsolete.

Recent advances in embryonic germ cell culture and in vitro differentiation could tap into a plentiful and economical source of germ-cells for both TN:ROSI and TN:ICSI transgenesis approaches (Marh J, et al., Biol Reprod 69: 169-176 (2003); Geijsen N, et al., Nature 427: 148-154 (2004)), leading to the safe and effective gene delivery in mammals. Extrapolating transposome transgenesis methodology from mice to larger animals would represent a significant improvement in technical ease and effectiveness. Such increases in effectiveness would reduce costs and, when extended to the livestock industry, may significantly facilitate the production of value added to commercial animals.

The teachings of all references, patents, and patent applications cited are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method for obtaining a transgenic embryo comprising:
a) incubating a mixture of a transposable exogenous nucleic acid and a transposase or a hyperactive mutant of said transposase or a nucleic acid encoding said transposase;
b) contacting said mixture with a sperm; and
c) introducing said mixture contacted with said sperm into an unfertilized oocyte to form a transgenic embryo;
whereby said transposase catalyzes integration of said transposable exogenous nucleic acid into the genome of said embryo.

2. The method of claim 1, wherein said transposable exogenous nucleic acid is a nucleic acid sequence flanked by at least two 19 base pair end sequences to form an inverted repeat that is recognized by said transposase.

3. The method of claim 2, wherein said end sequences are selected from a group consisting of SEQ ID NO: 1, SEQ ID NO: 2, and SEQ ID NO: 3.

4. The method of claim 1, wherein said exogenous nucleic acid is derived from a group consisting of vertebrates, invertebrates, and plants.

5. The method of claim 1, wherein said exogenous nucleic acid is derived from a group consisting of mammals, fish, amphibians, reptiles, and birds.

6. The method of claim 1, wherein said exogenous nucleic acid is derived from a group consisting of rodents, cows, pigs, sheep, goats, and horses.

7. The method of claim 1, wherein said exogenous nucleic acid contains at least one transgene.

8. The method of claim 1, wherein said exogenous nucleic acid contains more than one transposable exogenous sequence.

9. The method of claim 1, wherein said transposase is a prokaryotic or an eukaryotic transposase.

10. The method of claim 1, wherein said transposase is a hyperactive Tn5 transposase mutant.

11. The method of claim 1, wherein said transposase comprises an amino acid sequence having at least 80% identity to SEQ ID NO: 4.

12. The method of claim 1, wherein said transposase comprises SEQ ID NO: 4.

13. The method of claim 1, wherein said transposase is encoded by a nucleic acid sequence comprising SEQ ID NO: 5 or a codon biased nucleic acid sequence of said transposase.

14. The method of claim 1, wherein said transposase is encoded by a nucleic acid sequence comprising SEQ ID NO: 6.

15. The method of claim 3, wherein said end sequences are recognized by a Tn5 transposase or a hyperactive mutant of said transposase.

16. The method of claim 1, wherein said incubating is for about 5 minutes to about 5 hours.

17. The method of claim 1, wherein said incubating is for about 20 minutes to about 2 hours.

18. The method of claim 1, wherein said incubating is for about 30 minutes.

19. The method of claim 1, wherein said sperm is selected from a group consisting of a spermatozoon, a sperm head, and a spermatid.

20. The method of claim 1, wherein said sperm is a sperm head.

21. The method of claim 1, wherein said contacting is for about 30 seconds to about 5 minutes.

22. The method of claim 1, wherein said contacting is for about 2 minutes.

23. The method of claim 1, wherein said oocyte is an unfertilized metaphase II oocyte.

24. The method of claim 1, wherein said oocyte is selected from a group consisting of vertebrates, invertebrates, and plants.

25. The method of claim 1, wherein said oocyte is selected from a group consisting of rodents, cows, pigs, sheep, goats, fish, and horses.

26. The method of claim 1, wherein said mixture contacted with said sperm is introduced into said oocyte by microinjection.

27. The method of claim 1, wherein said embryo is implanted into a surrogate mother and develops into a transgenic non-human animal or plant.

28. A method for obtaining a transgenic embryo comprising:
a) incubating a mixture of a transposable exogenous nucleic acid and a transposase or a hyperactive mutant of said transposase or a nucleic acid encoding said transposase for about 30 minutes, wherein said exogenous nucleic acid is flanked by at least one inverted repeat of a nucleic acid sequence comprising SEQ ID NO: 1 and said transposase is a hyperactive Tn5 transposase mutant comprising an amino acid sequence of SEQ ID NO: 4 or encoded by a nucleic acid sequence comprising SEQ ID NO: 5 or a codon biased nucleic acid sequence comprising SEQ ID NO: 6;
b) contacting said mixture with a sperm head for about 2 minutes; and
c) introducing said mixture contacted with said sperm head into a metaphase II (MII) oocyte by microinjection to form a transgenic embryo;
whereby said transposase catalyzes integration of said transposable exogenous nucleic acid into the genome of said embryo.

29. The method of claim 28, wherein said exogenous nucleic acid is derived from a group consisting of vertebrates, invertebrates, and plants.

30. The method of claim 28, wherein said exogenous nucleic acid is derived from a group consisting of rodents, cows, pigs, sheep, goats, fish, and horses.

31. The method of claim 28, wherein said oocyte is selected from a group consisting of vertebrates, invertebrates, and plants.

32. The method of claim 28, wherein said oocyte is selected from a group consisting of rodents, cows, pigs, sheep, goats, fish, and horses.

33. The method of claim 28, wherein said transgenic embryo is implanted into a surrogate mother and develops into a transgenic animal or plant.

34. A method for generating a transgenic embryo comprising:
a) incubating a mixture of a transposable exogenous nucleic acid and a transposase or a hyperactive mutant of said transposase or a nucleic acid encoding said transposase; and
b) introducing said mixture into an in vitro fertilized (IVF) oocyte to form a transgenic embryo;
whereby said transposase catalyzes integration of said transposable exogenous nucleic acid into the genome of said embryo.

35. The method of claim 34, wherein said mixture is introduced into said IVF oocyte using a method selected from a group consisting of microinjection, electroporation, liposome vesicles, viral infection, and particle bombardment.

36. The method of claim 34, wherein said transposase is a prokaryotic or an eukaryotic transposase.

37. The method of claim 34, wherein said transposable exogenous nucleic acid is a nucleic acid sequence flanked by at least two 19 base pair end sequences to form an inverted repeat that is recognized by said transposase.

38. The method of claim 37, wherein said end sequences are selected from a group consisting of SEQ ID NO: 1, SEQ ID NO: 2, and SEQ ID NO: 3.

39. The method of claim 34, wherein said exogenous nucleic acid is derived from a group consisting of vertebrates, invertebrates, and plants.

40. The method of claim 34, wherein said exogenous nucleic acid is derived from a group consisting of mammals, fish, amphibians, reptiles, and birds.

41. The method of claim 34, wherein said exogenous nucleic acid is derived from a group consisting of rodents, cows, pigs, sheep, goats, and horses.

42. The method of claim 34, wherein said exogenous nucleic acid contains at least one transgene.

43. The method of claim 34, wherein said exogenous nucleic acid contains more than one transposable exogenous sequence.

44. The method of claim 34, wherein said transposase is a hyperactive Tn5 transposase mutant.

45. The method of claim 34, wherein said transposase comprises an amino acid sequence having at least 80% identity to SEQ ID NO: 4.

46. The method of claim 34, wherein said transposase comprises SEQ ID NO: 4.

47. The method of claim 34, wherein said transposase is encoded by a nucleic acid sequence comprising SEQ ID NO: 5 or a codon biased nucleic acid sequence of said transposase.

48. The method of claim 34, wherein said transposase is encoded by a nucleic acid sequence comprising SEQ ID NO: 6.

49. The method of claim 34, wherein said end sequences are recognized by a Tn5 transposase or a hyperactive mutant of said transposase.

50. The method of claim 34, wherein said incubating is for about 5 minutes to about 5 hours.

51. The method of claim 34, wherein said incubating is for about 20 minutes to about 2 hours.

52. The method of claim 34, wherein said incubating is for about 30 minutes.

53. The method of claim 34, wherein said IVF oocyte is selected from a group consisting of vertebrates, invertebrates, and plants.

54. The method of claim 34, wherein said IVF oocyte is selected from a group consisting of rodents, cows, pigs, sheep, goats, fish, and horses.

55. The method of claim 34, wherein said transgenic embryo is implanted into a surrogate mother and develops into a transgenic animal or plant.

56. A method for generating a transgenic embryo comprising:
a) incubating a mixture of a transposable exogenous nucleic acid and a transposase or a hyperactive mutant of said transposase or a nucleic acid encoding said transposase for about 30 minutes, wherein said exogenous nucleic acid is flanked by at least one inverted repeat of a nucleic acid sequence comprising SEQ ID NO: 1, SEQ ID NO: 2, or SEQ ID NO: 3 and said transposase is a hyperactive Tn5 transposase mutant comprising an amino acid sequence of SEQ ID NO: 4 or encoded by a nucleic acid sequence comprising SEQ ID NO: 5 or a codon biased nucleic acid sequence comprising SEQ ID NO: 6; and
b) introducing said mixture into an in vitro fertilized (IVF) oocyte by microinjection to form a transgenic embryo;
whereby said transposase catalyzes integration of said transposable exogenous nucleic acid into the genome of said embryo.

57. A method for obtaining a transgenic embryo comprising;
a) incubating a mixture of a transposable exogenous nucleic acid and a transposase or a hyperactive mutant of said transposase or a nucleic acid encoding said transposase;
b) contacting said mixture with a round spermatid; and
c) introducing said mixture contacted with said spermatid -into an artificially activated oocyte to form a transgenic embryo;
whereby said transposase catalyzes integration of said transposable exogenous nucleic acid into the genome of said embryo.

58. The method of claim 57, wherein said transposable exogenous nucleic acid is a nucleic acid sequence flanked by at least two 19 base pair end sequences to form an inverted repeat that is recognized by said transposase.

59. The method of claim 58, wherein said end sequences are selected from a group consisting of SEQ ID NO: 1, SEQ ID NO: 2, and SEQ ID NO: 3.

60. The method of claim 57, wherein said exogenous nucleic acid is derived from a group consisting of vertebrates, invertebrates, and plants.

61. The method of claim 57, wherein said exogenous nucleic acid is derived from a group consisting of mammals, fish, amphibians, reptiles, and birds.

62. The method of claim 57, wherein said exogenous nucleic acid is derived from a group consisting of rodents, cows, pigs, sheep, goats, and horses.

63. The method of claim 57, wherein said exogenous nucleic acid contains at least one transgene.

64. The method of claim 57, wherein said exogenous nucleic acid contains more than one transposable exogenous sequence.

65. The method of claim 57, wherein said transposase is a prokaryotic or an eukaryotic transposase.

66. The method of claim 57, wherein said transposase is a hyperactive Tn5 transposase mutant.

67. The method of claim 57, wherein said transposase comprises an amino acid sequence having at least 80% identity to SEQ ID NO: 4.

68. The method of claim 57, wherein said transposase comprises SEQ ID NO: 4.

69. The method of claim 57, wherein said transposase is encoded by a nucleic acid sequence comprising SEQ ID NO: 5 or a codon biased nucleic acid sequence of said transposase.

70. The method of claim 57, wherein said transposase is encoded by a nucleic acid sequence comprising SEQ ID NO: 6.

71. The method of claim 59, wherein said end sequences are recognized by a Tn5 transposase or a hyperactive mutant of said transposase.

72. The method of claim 57, wherein said incubating is for about 5 minutes to about 5 hours.

73. The method of claim 57, wherein said incubating is for about 20 minutes to about 2 hours.

74. The method of claim 57, wherein said incubating is for about 30 minutes.

75. The method of claim 57, wherein said contacting is for about 30 seconds to about 5 minutes.

76. The method of claim 57, wherein said contacting is for about 2 minutes.

77. The method of claim 57, wherein said oocyte is an unfertilized metaphase II oocyte.

78. The method of claim 57, wherein said oocyte is selected from a group consisting of vertebrates, invertebrates, and plants.

79. The method of claim 57, wherein said oocyte is selected from a group consisting of rodents, cows, pigs, sheep, goats, fish, and horses.

80. The method of claim 57, wherein said transgenic embryo is implanted into a surrogate mother and develops into a transgenic animal or plant.

81. A method for obtaining a transgenic embryo comprising:
a) incubating a mixture of a transposable exogenous nucleic acid and a transposase or a hyperactive mutant of said transposase or a nucleic acid encoding said transposase for about 30 minutes, wherein said exogenous nucleic acid is flanked by at least one inverted repeat of a nucleic acid sequence comprising SEQ ID NO: 1, SEQ ID NO: 2, or SEQ ID NO: 3 and said transposase is a hyperactive Tn5 transposase mutant comprising an amino acid sequence of SEQ ID NO: 4 or transposase mutant comprising an amino acid sequence of SEQ ID NO: 4 or encoded by a nucleic acid sequence comprising SEQ ID NO: 5 or a codon biased nucleic acid sequence comprising SEQ ID NO: 6;
b) contacting said mixture with a round spermatid for about 2 minutes; and
c) introducing said mixture contacted with said spermatid into an artificially activated ocyte by microinjection to form a transgenic embryo;
whereby said transposase catalyzes integration of said transposable exogenous nucleic acid into the genome of said embryo.

82. A method for obtaining a cloned transgenic embryo comprising:
a) removing a nucleus from an oocyte to form an anucleated oocyte;
b) incubating a mixture of a transposable exogenous nucleic acid and a transposase or a hyperactive mutant of said transposase or a nucleic acid encoding said transposase;
c) introducing said mixture and an exogenous diploid nucleus into said anucleated oocyte to form a nucleated oocyte; and
d) activating said nucleated oocyte to form a cloned transgenic embryo;
whereby said transposase catalyzes integration of said transposable exogenous nucleic acid into the genome of said cloned transgenic embryo.
